(19) United States
(12) Patent Application Publication
(10) Pub. No.: US 2024/0139412 A1
(43) Pub. Date: May 2, 2024

(54) VASCULAR PUNCTURE DEVICE AND VASCULAR PUNCTURE SYSTEM

(71) Applicant: TERUMO KABUSHIKI KAISHA, Tokyo (JP)

(72) Inventors: Takito INUKAI, Isehara-shi (JP); Takumi FUKUDA, Hadano-shi (JP); Yoichiro KUWANO, Tokyo (JP)

(73) Assignee: TERUMOKABUSHIKI KAISHA, Tokyo (JP)

(21) Appl. No.: 18/410,346

(22) Filed: Jan. 11, 2024

Related U.S. Application Data
(63) Continuation of application No. PCT/JP2022/027827, filed on Jul. 15, 2022.

(30) Foreign Application Priority Data
Jul. 21, 2021 (JP) 2021-120220

(51) Int. Cl. A61M 5/158; G16H 40/63

(52) U.S. Cl. CPC A61M 5/158; A61M 2005/1585; A61M 2005/1588; A61M 2205/10; A61M 2205/13; A61M 2205/3327; A61M 2210/005; A61M 2230/005; G16H 40/63

(57) ABSTRACT

A vascular puncture device is a vascular puncture device that punctures a blood vessel using an inner needle having a sharp needle tip, a flexible outer tube covering the inner needle, a drive unit moving the inner needle and the outer tube, and an imaging unit capable of acquiring a cross-sectional image of a human body in contact with a skin surface and visualizing a distal end of the inner needle, and includes a control unit controlling movement of the drive unit. The control unit calculates one or more of a movement distance and a puncture angle of the drive unit based on a separation distance between the distal end of the inner needle and a distal end of the outer tube, and moves the drive unit to set the drive unit at one or more of the calculated movement distance and the calculated puncture angle.

### CROSS-REFERENCES TO RELATED APPLICATIONS

This application is a continuation of International Application No. PCT/JP2022/027827 filed on Jul. 15, 2022, which claims priority to Japanese Application No. 2021-120220 filed on Jul. 21, 2021, the entire content of both of which is incorporated herein by reference.

### TECHNOLOGICAL FIELD

The present invention relates to a vascular puncture device and a vascular puncture system capable of detecting and puncturing a position of a blood vessel from an image acquired by an echographic device.

### BACKGROUND DISCUSSION

In order to secure an access route to a blood vessel for drug administration and endovascular treatment, vascular puncture is performed in which a human body is punctured with an inner needle having a sharp needle tip, the inner needle being covered by a flexible outer tube. The access route can be secured due to the outer tube since only the inner needle is removed after the inner needle and the outer tube reach the inside of the blood vessel. In the vascular puncture, an operator cannot visually observe the blood vessel from a skin surface, and thus, a position of the blood vessel is estimated by standard knowledge of blood vessel running and skill such as tactile perception of blood vessel pulsation.

In recent years, there is a device that identifies a position of a blood vessel by a sensor, determines a puncture angle and a puncture path from a shape of the blood vessel or the like, and automatically performs vascular puncture by a robot arm (see, for example, U.S. Pat. No. 9,364,171).

By the way, for example, puncture of a radial artery is currently performed using a method in which a position of a blood vessel is identified based on the visual sense and tactile sense of an operator and a position of a needle tip with respect to the blood vessel is identified based on the presence or absence of backflow of blood from an inner needle. A method of sticking a needle into both a front wall and a back wall of a blood vessel, then retracting the needle, and removing the needle from the back wall, that is, so-called double wall puncture (DWP) is widely used in order to increase a success rate of the puncture and reduce the number of trials. Compared to a method of sticking an inner needle only into a front wall of a blood vessel, that is, so-called single wall puncture (SWP), the DWP has no difference in terms of bleeding and occurrence of radial artery occlusion (RAO).

When the vascular puncture is automatically performed, skill such as tactile perception of an operator cannot be used in order to obtain a state where a distal end of an outer tube is disposed in the blood vessel. Therefore, when the vascular puncture is automatically performed, it is difficult to dispose the distal end of the outer tube in the blood vessel in an appropriate state. For example, in a case where the distal end of the outer tube is located near a blood vessel wall of the blood vessel close to the skin, the distal end of the outer tube may come out of the blood vessel when the inner needle is removed. In a case where the distal end of the outer tube moves out of the blood vessel, it is necessary to perform puncture again, and the burden on a patient increases.

### SUMMARY

A vascular puncture device and a vascular puncture system are disclosed, which are capable of appropriately disposing a distal end of an outer tube, which covers an inner needle, inside a blood vessel when vascular puncture is automatically performed.

A vascular puncture device is disclosed that punctures a blood vessel using an inner needle including a needle tip that is sharp, an outer tube that is flexible and covers the inner needle, a drive unit that moves the inner needle and the outer tube, and an imaging unit capable of acquiring a cross-sectional image of a human body in contact with a skin surface and visualizing a distal end of the inner needle, and includes a control unit capable of controlling movement of the drive unit. The control unit calculates one or more of a movement distance and a puncture angle of the drive unit based on information on the cross-sectional image and a separation distance between the distal end of the inner needle and a distal end of the outer tube, and moves the drive unit to set the drive unit at one or more of the calculated movement distance and the calculated puncture angle.

A vascular puncture system is disclosed, which includes: an inner needle including a needle tip that is sharp; an outer tube that is flexible and covers the inner needle; a drive unit that moves the inner needle and the outer tube; an imaging unit capable of acquiring a cross-sectional image of a human body in contact with a skin surface and visualizing a distal end of the inner needle; and a control unit capable of controlling movement of the drive unit. The control unit calculates one or more of a movement distance and a puncture angle of the drive unit based on the information on the cross-sectional image and a separation distance between the distal end of the inner needle and the distal end of the outer tube, and moves the drive unit so that the drive unit becomes one or more of the calculated movement distance and the calculated puncture angle.

In the vascular puncture device and the vascular puncture system configured as described above, a position of the distal end of the outer tube can be calculated by the control unit when vascular puncture is automatically performed by the inner needle, and thus, the distal end of the outer tube covering the inner needle can be appropriately disposed in the blood vessel.

A method is disclosed for puncturing a blood vessel, the method comprising: acquiring a cross-sectional image of a human body in contact with a skin surface and visualizing a distal end of an inner needle, the inner needle including a needle tip; calculating one or more of a movement distance and a puncture angle based on information on the cross-sectional image and a separation distance between the distal end of the inner needle and a distal end of an outer tube, the outer tube covering the inner needle; and setting a drive unit at one or more of the calculated movement distance and the calculated puncture angle, the drive unit configured to move the inner needle and the outer tube.

### BRIEF DESCRIPTION OF THE DRAWINGS

FIG. 1 is a side view of a vascular puncture system according to the present embodiment.

FIG. 2 is a top view of the vascular puncture system, which illustrates a positional relationship with an arm whose cross-sectional image is to be acquired.

FIG. 3 is a configuration diagram of the vascular puncture system.

FIG. 4 is a view illustrating an example of an image acquired by an imaging unit.

FIG. 5 is a side view illustrating the vascular puncture system immediately before puncture in a state where a probe is inclined with respect to a skin surface.

FIG. 6 is a top view illustrating the vascular puncture system immediately before the puncture in the state where the probe is inclined with respect to the skin surface.

FIG. 7 is a side view illustrating the vascular puncture system immediately after the puncture in the state where the probe is inclined with respect to the skin surface.

FIG. 8 is a flowchart illustrating a flow of control in a control unit.

### DETAILED DESCRIPTION

Set forth below with reference to the accompanying drawings is a detailed description of embodiments of a vascular puncture device and a vascular puncture system capable of detecting and puncturing a position of a blood vessel from an image acquired by an echographic device. Note that dimensional ratios in the drawings may be exaggerated and different from actual ratios for convenience of description.

A vascular puncture system 10 according to the embodiment of the present disclosure is used when puncturing an arm H of a human body to acquire a cross-sectional image of the arm H, detect a position of an artery to be punctured, and automatically puncture the artery.

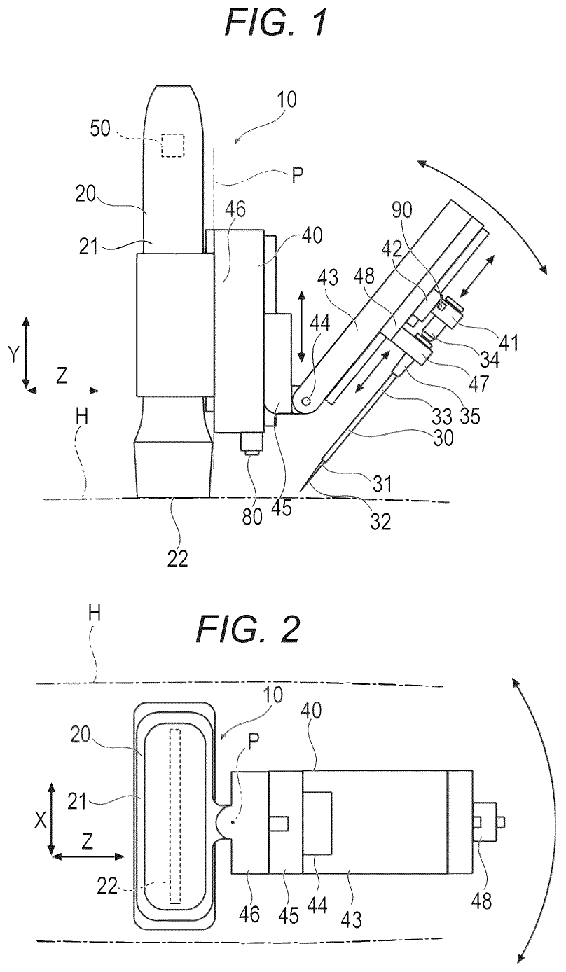

As illustrated in FIGS. 1 to 3, the vascular puncture system 10 includes: a probe 20 having an imaging unit 22 that comes into contact with a skin surface and acquires a cross-sectional image of a human body; a puncture unit 30 that performs puncture; a drive unit 40 that moves the puncture unit 30 with respect to the probe 20; and a vascular puncture device 11. The vascular puncture device 11 includes an inclination detection unit 50 that detects an inclination angle of the probe 20, a control unit 60 that performs image analysis of the cross-sectional image and control of the drive unit 40, and a display unit 70 capable of displaying the cross-sectional image. Further, the vascular puncture device 11 includes a detection unit 80 that detects a position of a distal end of the puncture unit 30 before puncture, and a reaction force measurement unit 90 that detects a reaction force acting on the puncture unit 30.

The probe 20 includes a vertically elongated handle portion 21 gripped by an operator, an imaging unit 22 disposed at a lower end of the handle portion 21, a transmitter 23 that transmits a signal from the control unit 60 to the imaging unit 22, and a receiver 24 that transmits a signal from the imaging unit 22 to the control unit 60.

The imaging unit 22 is provided so as to extend over substantially the entire width at the central portion of a lower surface of the probe 20. The imaging unit 22 can be an echographic device that includes a transducer that generates an ultrasound wave and obtain the cross-sectional image of the inside of the human body by detecting a reflected wave of the ultrasound wave. In the present embodiment, the cross-sectional image orthogonal to an axial direction of the blood vessel is acquired, and thus, the imaging unit 22 is disposed such that a length direction of the imaging unit 22 is orthogonal to a length direction of the arm H.

The transmitter 23 transmits a signal from the control unit 60 to the imaging unit 22 in order to output an ultrasound wave from the imaging unit 22. The receiver 24 transmits, to the control unit 60, a signal output from the imaging unit 22 receiving the reflected wave.

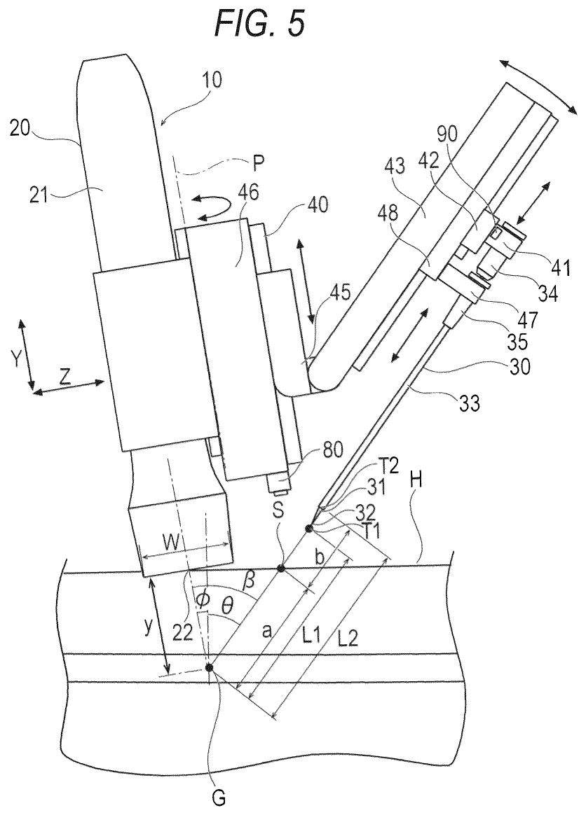

The inclination detection unit 50 is connected to the control unit 60. The inclination detection unit 50 can be, for example, a gyro sensor, and can detect an inclination of the probe 20. A reference of the inclination is a perpendicular direction orthogonal to the horizontal direction. Since an upper surface of the arm H when performing the puncture is set along the horizontal direction, an inclination of the vascular puncture system 10 with respect to a perpendicular line of the skin surface can be detected by detecting the inclination with respect to the perpendicular direction by the inclination detection unit 50. In the present example, it is assumed that the inclination detection unit 50 detects that the vascular puncture system 10 is inclined at an angle of φ as illustrated in FIG. 5. The inclination detection unit 50 is not limited to the gyro sensor, and may be, for example, a camera that captures an image of the skin surface of the arm H. In this case, the control unit 60 can detect the inclination φ of the probe 20 from a result of the image capturing by the inclination detection unit 50 using a technique such as machine learning or deep learning. In addition, the inclination detection unit 50 is not necessarily provided.

As illustrated in FIGS. 1 and 5, the puncture unit 30 can include an inner needle 31 made of metal and having a sharp needle tip 32 formed at a distal end, and a flexible tubular outer tube 33 disposed so as to cover an outer peripheral surface of the inner needle 31. The inner needle 31 may be either hollow or solid. If the inner needle 31 is hollow, a position of the needle tip 32 with respect to a blood vessel can be identified by the presence or absence of backflow of blood from the inner needle 31. If the inner needle 31 is solid, the outer diameter of the inner needle 31 can be reduced in a case where the inner needle 31 has rigidity similar to that of a hollow needle. Therefore, a puncture hole formed by the inner needle 31 can be reduced, bleeding can be reduced, and burden on a patient can be reduced.

The needle tip 32 is a portion having a blade surface inclined with respect to an axial center on a side closer to the distal end than a portion where the outer diameter of the inner needle 31 is constant. Alternatively, the needle tip 32 may be a portion whose outer diameter decreases toward the most distal end that is sharp.

As illustrated in FIGS. 1 and 5, the needle tip 32 protrudes from the outer tube 33 in a state where the outer tube 33 covers the outer side of the inner needle 31. An inner needle hub 34 is fixed to a proximal end portion of the inner needle 31. A tubular outer tube hub 35 is fixed to a proximal end portion of the outer tube 33. A distal end portion of the inner needle hub 34 can be tapered in diameter toward the distal end, and can be tapered and fitted to a tapered opening on a proximal end side of the outer tube hub 35.

As illustrated in FIGS. 1 and 2, the drive unit 40 includes: a first holding portion 41 that holds the inner needle hub 34; a first linear movement portion 42 that linearly moves the first holding portion 41; a second holding portion 47 that holds the outer tube hub 35; a second linear movement portion 48 that linearly moves the second holding portion 47; an inclination portion 43 that inclines the first holding portion 41 and the second holding portion 47; a third linear movement portion 45 that moves the inclination portion 43 in a length direction of the probe 20; and a rotation portion 46 that rotates the third linear movement portion 45 about a predetermined rotation axis P.

The first holding portion 41 can detachably hold the inner needle hub 34. The first holding portion 41 can be, for example, a clamp that can perform holding so as to sandwich the inner needle hub 34.

The first linear movement portion 42 can linearly move the first holding portion 41 holding the inner needle hub 34 of the puncture unit 30 forward and backward along an extending direction (puncture direction) of the inner needle 31. The first linear movement portion 42 is used to change a separation distance of the inner needle 31 from the outer tube 33. The first linear movement portion 42 can include, for example, a rotational drive source such as a motor whose driving can be controlled by the control unit 60, and a structure (for example, a feed screw mechanism) that converts a rotational motion of the rotational drive source into a linear motion.

The second holding portion 47 can detachably hold the outer tube hub 35. The second holding portion 47 can be, for example, a clamp that can perform holding so as to sandwich the outer tube hub 35.

The second linear movement portion 48 can linearly move the first linear movement portion 42 holding the inner needle hub 34 via the first holding portion 41 and the second holding portion 47 holding the outer tube hub 35 forward and backward along the extending direction (puncture direction) of the inner needle 31 and the outer tube 33. That is, the second linear movement portion 48 can integrally move the outer tube 33 and the inner needle 31 at the time of puncture with the inner needle 31. The second linear movement portion 48 can include, for example, a rotational drive source such as a motor whose driving can be controlled by the control unit 60, and a structure (for example, a feed screw mechanism) that converts a rotational motion of the rotational drive source into a linear motion.

The inclination portion 43 can incline the second linear movement portion 48. The inclination portion 43 is used to change puncture angles of the inner needle 31 and the outer tube 33 with respect to a surface of a skin of the patient. The inclination portion 43 can include a hinge 44 whose angle can be changed, and a rotational drive source such as a motor whose driving can be controlled by the control unit 60 in order to change the angle of the hinge 44.

The third linear movement portion 45 is used to bring the puncture unit 30 close to (i.e., towards) or away from the skin of the patient. The third linear movement portion 45 can linearly move the inclination portion 43 forward and backward along an extending direction of the probe 20. The third linear movement portion 45 can include, for example, a rotational drive source such as a motor whose driving can be controlled by the control unit 60, and a structure (for example, a feed screw mechanism) that converts a rotational motion of the rotational drive source into a linear motion.

The rotation portion 46 is used to change a direction of the inner needle 31 when the third linear movement portion 45 is viewed substantially perpendicularly to the surface of the skin of the patient. The rotation portion 46 can rotate the inclination portion 43 about the rotation axis P parallel to the length direction of the probe 20. The rotation portion 46 can include, for example, a rotational drive source such as a motor whose driving can be controlled by the control unit 60.

The detection unit 80 is used to identify relative positions of the distal end of the inner needle 31 and a distal end of the outer tube 33 with respect to the imaging unit 22 before puncture. For example, the detection unit 80 is fixed to the probe 20 at an accurately defined position with respect to the imaging unit 22, and can capture images of the distal end of the inner needle 31 and the distal end of the outer tube 33 before puncture. The detection unit 80 is not necessarily a camera as long as the relative positions of the distal end of the inner needle 31 and the distal end of the outer tube 33 with respect to the imaging unit 22 before puncture can be identified, and may be, for example, a reflected light proximity sensor or an outer diameter measuring device.

The reaction force measurement unit 90 is used to measure a reaction force in the puncture direction acting on the inner needle 31 at the time of puncture. The reaction force measurement unit 90 can be disposed, for example, in the first holding portion 41, but a place where the reaction force measurement unit is disposed is not limited as long as the force can be detected. The reaction force measurement unit 90 may be disposed, for example, in the second holding portion 47. The reaction force measurement unit 90 transmits a detected signal to the control unit 60.

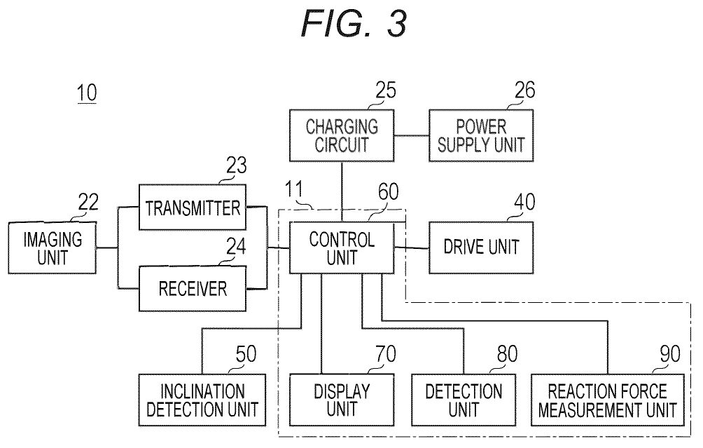

As illustrated in FIGS. 1 and 3, the control unit 60 transmits a signal to the imaging unit 22 via the transmitter 23 and causes the imaging unit 22 to output an ultrasound wave. In addition, the control unit 60 can form a cross-sectional image from a signal obtained from the imaging unit 22 via the receiver 24. Further, the control unit 60 can cause the display unit 70 to display the obtained cross-sectional image. Further, the control unit 60 can receive information on a separation distance between the distal end of the inner needle 31 and the distal end of the outer tube 33 from the detection unit 80, perform arithmetic processing such as image analysis from information on the cross-sectional image, and control the operation of the drive unit 40 based on the result. Further, the control unit 60 can receive information on a reaction force acting on the inner needle 31 from the reaction force measurement unit 90, perform arithmetic processing, and control the operation of the drive unit 40 based on the result. The control unit 60 can include, as physical configurations, a storage circuit and an arithmetic circuit. The storage circuit can store programs and various parameters. The arithmetic circuit can perform arithmetic processing.

The control unit 60 can be connected to a power supply unit 26 including, for example, a rechargeable battery via a charging circuit 25. In addition, the control unit 60 is connected to the inclination detection unit 50. The control unit 60 may be disposed in the probe 20 or the drive unit 40, or may be configured separately from the probe 20 or the drive unit 40.

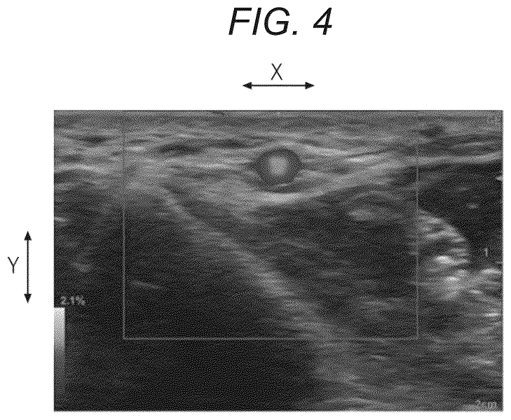

The control unit 60 acquires a cross-sectional image as illustrated in FIG. 4 from the imaging unit 22. It is assumed that a lateral direction in the cross-sectional image, that is, a width direction of the arm H is an X direction, a longitudinal direction in the cross-sectional image, that is, a depth direction of the arm H is a Y direction, and a direction orthogonal to the paper surface of the cross-sectional image, that is, the length direction of the arm H is a Z direction. Coordinates of an upper left point in the cross-sectional image are set as a start point (0, 0, 0).

The control unit 60 can identify a position of the blood vessel in the image by performing image analysis of the acquired cross-sectional image. In addition, the control unit 60 can detect contact of the inner needle 31 with a blood vessel wall by analyzing a measurement result from the reaction force measurement unit 90. In addition, the control unit 60 can detect the relative positions of the distal end of the inner needle 31 and the distal end of the outer tube 33 with respect to the imaging unit 22 with respect to the imaging unit 22 by performing image analysis on a detection result from the detection unit 80. The analysis and control in the control unit 60 will be described in detail later.

As illustrated in FIGS. 3 and 4, the display unit 70 is a monitor or the like capable of displaying the cross-sectional image. The display unit 70 can calculate a position of the distal end of the outer tube 33 whose image is hardly captured (i.e., difficult to detect) by the imaging unit 22, which utilizes an ultrasound wave, by the control unit 60 based on the detection result from the detection unit 80, and display the position to be overlaid on the cross-sectional image.

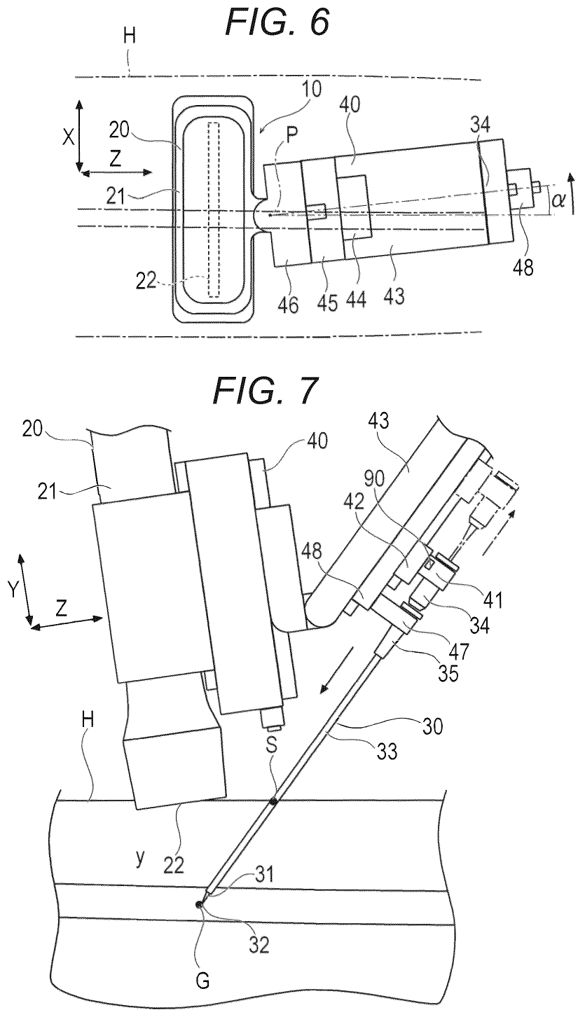

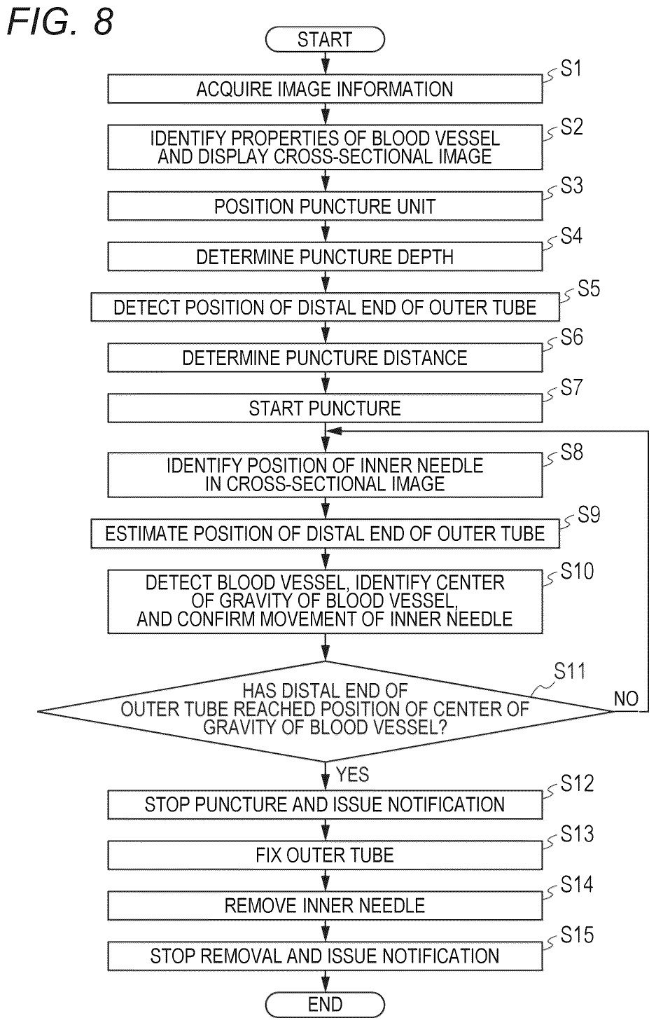

Next, a method of puncturing a blood vessel using the vascular puncture system 10 will be described with reference to a flowchart of the control unit 60 illustrated in FIG. 8. As illustrated in FIGS. 1 and 2, the vascular puncture system 10 is used in contact with the skin surface as illustrated in FIGS. 5 and 6.

The control unit 60 acquires image information from the imaging unit 22 via the receiver 24 (S1). The control unit 60 forms a cross-sectional image from the image information. The control unit 60 identifies a position of the blood vessel, the center of gravity of the blood vessel, the blood vessel wall, and the like in the image by performing image analysis on the obtained cross-sectional image, and causes the display unit 70 to display the cross-sectional image (S2). The cross-sectional image is continuously displayed on the display unit 70 until a procedure is completed while being updated to new data in substantially real time. In order to identify the position of the blood vessel, the center of gravity of the blood vessel, a blood vessel wall, and the like in the image, the control unit 60 can prepare a large number of images of the same type and use a technique such as machine learning or deep learning. In addition, it is also possible to detect a region with blood flow by the Doppler method in the imaging unit 22 and recognize the region as a region of the blood vessel.

The control unit 60 sets a position G of the center of gravity of the identified region recognized as the blood vessel in the image as the position of the blood vessel. Coordinates of the detected position G of the center of gravity of the blood vessel are defined as (x, y, 0). Next, the control unit 60 calculates a position (coordinates) and a posture (angle) of the puncture unit 30 desired for puncture, and positions the puncture unit 30 so as to be at the position with the posture (S3). In the present embodiment, the control unit 60 calculates, for example, an inner needle preparation position T1, a puncture angle θ, and a rotation angle α. The inner needle preparation position T1 is a position of the needle tip 32 immediately before puncture. The puncture angle θ is an angle at which the inner needle 31 at the time of puncture is inclined with respect to a perpendicular line of the skin surface. The rotation angle α is an angle at which the inner needle 31 at the time of puncture is inclined with respect to the Z direction when the surface of the arm H is viewed from a direction of the perpendicular line. The puncture angle θ can also be, for example, a preset angle (for example, 30 degrees). The rotation angle α is set within a range in which the needle tip 32 of the inner needle 31 can reach the inside of an artery. The inner needle preparation position T1 is set at a certain height from the surface of the skin. The inner needle preparation position T1 is a position where the inner needle 31 can reach the inside of the blood vessel on the cross-sectional image by being caused to protrude along the extending direction (puncture direction).

The control unit 60 first acquires a cross-sectional image from the imaging unit 22. In the cross-sectional image, the Y direction is inclined at the angle of φ with respect to the perpendicular line of the skin surface. In addition, the control unit 60 acquires the inclination φ of the vascular puncture system 10 by the inclination detection unit 50. The control unit 60 sets an upper left end position of the acquired cross-sectional image as a start point (0, 0, 0). With this start point as a reference, the control unit 60 detects the position G of the center of gravity of each blood vessel from the cross-sectional image.

For example, coordinates of the position G of the center of gravity of the detected blood vessel are set to (x, y, 0), and the rotation angle α can be set to 0 degrees. A coordinate y1 in the Y direction of a puncture position S on the skin surface can be calculated by y1=y−a·cos(φ+θ) as illustrated in FIG. 5. A coordinate z1 in the Z direction of the puncture position S can be calculated by z1=a·sin(φ+θ). In addition, a puncture depth a is calculated by a=y·cos φ/cos θ. As a result, coordinates (x, y1, z1) of the puncture position S and the puncture depth a are defined (S4).

Alternatively, the above-described puncture angle θ may be calculated from a width W of the probe 20. Assuming that the coordinate z1 of the puncture position S in the Z direction is half the width W of the probe 20, z1=W/2 is calculated. The puncture angle θ is calculated by θ=arctan ((z1−y·sin θ)/(y·cos φ)).

A distance L from the inner needle preparation position T1 where the needle tip 32 is disposed to the position G of the center of gravity is set to a value longer than the puncture depth a. An angle β between a plane of the cross-sectional image and the puncture direction is obtained by β=θ+φ, and coordinates of the inner needle preparation position T1 can be identified by defining a distance L1 from the position G of the center of gravity to the puncture position S, the rotation angle α, and the angle β. When the coordinates of the inner needle preparation position T1 are (x, y2, z2) and the rotation angle α is 0 degrees, the coordinate y2 in the Y direction can be calculated by y2=y−L·cos (φ+θ). The coordinate z2 in the Z direction can be calculated by z2=L·sin(φ+θ).

Next, the control unit 60 controls and drives at least one of the second linear movement portion 48, the third linear movement portion 45, the inclination portion 43, or the rotation portion 46 such that the inner needle 31 satisfies the puncture distance L2, the rotation angle α, and the angle β. As a result, the puncture unit 30 is positioned at the desired position with the desired posture. At this time, the distal end of the needle tip 32 of the inner needle 31 is disposed at the inner needle preparation position T1. In order to maintain a relative positional relationship between the inner needle 31 and the outer tube 33, it is preferable not to operate the first linear movement portion 42 in positioning before puncture.

Next, the control unit 60 calculates the position of the distal end of the inner needle 31 and the position of the distal end of the outer tube 33 from the detection result received from the detection unit 80 (S5). Since a position of the detection unit 80 with respect to the imaging unit 22 can be accurately defined, the control unit 60 can accurately identify coordinates of the inner needle preparation position T1 where the distal end of the inner needle 31 before puncture is located and coordinates of an outer tube preparation position T2 where the distal end of the outer tube 33 before puncture is located.

In a case where information on dimensions of the inner needle 31 and the outer tube 33 can be acquired or stored, the control unit 60 may identify the coordinates of the outer tube preparation position T2 from the information. The information on the dimensions of the inner needle 31 and the outer tube 33 can be, for example, a length of a blade surface of the needle tip 32, a length of the inner needle 31, a length of the outer tube 33, and a protruding height of the inner needle 31 from the outer tube 33 (a distance from the inner needle preparation position T1 to the outer tube preparation position T2).

Next, the control unit 60 calculates a distance b from the puncture position S to the outer tube preparation position T2. Subsequently, the control unit 60 determines a distance from the outer tube preparation position T2 to the position G of the center of gravity as a puncture distance L2=a+b (S6).

Next, the control unit 60 starts puncture of the identified blood vessel (S7). The control unit 60 receives an instruction to start the puncture from the operator by an input means such as a switch, a keyboard, or a mouse connected to the control unit 60. In response to this instruction, the control unit 60 drives the second linear movement portion 48. As a result, the inner needle 31 and the outer tube 33 integrally move in the puncture direction as illustrated in FIG. 7.

The control unit 60 performs image analysis on the cross-sectional image obtained from the imaging unit 22 and identifies a position of the inner needle 31 (S8). Then, the control unit 60 estimates the position of the distal end of the outer tube 33 from the obtained information on the position of the distal end of the inner needle 31 and information on the protruding height of the inner needle 31 detected by the detection unit 80 (the distance from the inner needle preparation position T1 to the outer tube preparation position T2) (S9). The control unit 60 performs image analysis on the cross-sectional image to identify a region of the blood vessel, and confirms the position G of the center of gravity of the blood vessel and the presence or absence of movement of the inner needle 31 (S10). In addition, the control unit 60 can also cause the display unit 70 to display an image in which the position G of the center of gravity of the blood vessel, the region of the blood vessel, the position of the distal end of the inner needle 31, the position of the distal end of the outer tube 33, and the like are overlaid on the cross-sectional image in real time.

Next, the control unit 60 determines whether the distal end of the outer tube 33 has reached the position G of the center of gravity of the blood vessel (S11). When determining that the distal end of the outer tube 33 has not reached the position G of the center of gravity of the blood vessel, the control unit 60 repeats the above-described steps S8 to S11 until determining that the distal end has reached the position G of the center of gravity.

When determining that the distal end of the outer tube 33 has reached the position G of the center of gravity of the blood vessel, the control unit 60 stops the second linear movement portion 48 to stop the puncture (S12). The control unit 60 causes the display unit 70 to display and notify that the puncture is stopped.

Next, the control unit 60 fixes the outer tube 33 so as not to move (S13). Since the second linear movement portion 48 capable of moving the outer tube 33 and the first linear movement portion 42 capable of moving the inner needle 31 independently of the outer tube 33 are provided in the present embodiment, the outer tube 33 can be fixed so as not to move by preventing the second linear movement portion 48 from being operated. Therefore, the second linear movement portion 48 corresponds to an outer tube holding unit. Note that, for example, in a case where only one linear movement portion for integrally moving the inner needle 31 and the outer tube 33 is provided in order to move the puncture unit 30 in the puncture direction, it is preferable to remove the outer tube 33 from the inner needle 31 so as to allow the outer tube 33 not to be affected by the movement of the linear movement portion. Then, the outer tube 33 removed from the inner needle 31 may be fixable by, for example, the outer tube holding unit such as a clamp provided in the inclination portion 43 or the like.

When the inner needle 31 and the outer tube 33 move integrally by the puncture distance L2 and the distal end of the outer tube 33 reaches the vicinity of the position G of the center of gravity of the blood vessel, the distal end of the inner needle 31 reaches the back of the position G of the center of gravity of the blood vessel.

The control unit 60 can detect contact of the inner needle 31 with the blood vessel from a reaction force received by the inner needle 31 detected by the reaction force measurement unit 90. The reaction force detected by the reaction force measurement unit 90 increases when the inner needle 31 punctures a wall portion (front wall) of the blood vessel on a side closer to the skin, and further increases when the inner needle 31 punctures a wall portion (back wall) of the blood vessel on a side farther from the skin. Therefore, the control unit 60 can detect that the reaction force measurement unit 90 has reached the front wall or the back wall of the blood vessel by monitoring a temporal change in the reaction force received from the reaction force measurement unit 90 and detecting an increase in the reaction force. Therefore, for example, the control unit 60 can detect that the distal end of the inner needle 31 has reached the back wall and stop the puncture as necessary.

Next, in a state where the second linear movement portion 48 that integrally moves the outer tube 33 and the inner needle 31 is stopped, the control unit 60 drives the first linear movement portion 42 that moves only the inner needle 31 to remove the inner needle 31 from the outer tube 33 (S14). After the removal of the inner needle 31 is completed, the control unit 60 stops the first linear movement portion 42 and completes the removal (S15). The control unit 60 causes the display unit to display and notify that the removal has been completed. As a result, the control by the control unit 60 is completed.

Note that steps S13 to 15 after fixing of the outer tube 33 may be manually performed.

After removing the inner needle 31 with the outer tube 33 being left, the operator inserts a guide wire from a proximal end opening of the outer tube hub 35 by a defined length. Subsequently, the operator removes the outer tube 33 with the guide wire being left, whereby a procedure for securing an access route to the blood vessel is completed.

As described above, the vascular puncture device 11 according to the present embodiment is the vascular puncture device 11 that punctures a blood vessel using the inner needle 31 having the sharp needle tip 32, the flexible outer tube 33 covering the inner needle 31, the drive unit 40 moving the inner needle 31 and the outer tube 33, and the imaging unit 22 capable of acquiring a cross-sectional image of a human body in contact with a skin surface and visualizing a distal end of the inner needle 31, and includes the control unit 60 capable of controlling movement of the drive unit 40. The control unit 60 calculates a movement distance and/or a puncture angle of the drive unit 40 based on information on the cross-sectional image and a separation distance between the distal end of the inner needle 31 and a distal end of the outer tube 33, and moves the drive unit 40 to set the drive unit 40 at the calculated movement distance and/or the calculated puncture angle.

In the vascular puncture device 11 configured as described above, a position of the distal end of the outer tube 33 can be calculated by the control unit 60 when vascular puncture is automatically performed by the inner needle 31, and thus, the distal end of the outer tube 33 covering the inner needle 31 can be appropriately disposed in the blood vessel. Therefore, when the inner needle 31 is removed, it is possible to suppress the distal end of the outer tube 33 from coming out of the blood vessel and to help reduce the burden on a patient during re-puncture.

In addition, the control unit 60 controls the drive unit 40 such that a position separated by the separation distance in a proximal direction from the distal end of the inner needle 31 along an axial center of the inner needle 31 coincides with the position G of the center of gravity of the blood vessel calculated from the information on the cross-sectional image when the puncture of the blood vessel is completed. As a result, the control unit 60 can appropriately dispose the distal end of the outer tube 33 at the position G of the center of gravity of the blood vessel. Therefore, when the inner needle 31 is removed, it is possible to suppress the distal end of the outer tube 33 from coming out of the blood vessel and to help reduce the burden on a patient during re-puncture. Note that the separation distance between the distal end of the inner needle 31 and the distal end of the outer tube 33 may be manually input by an input means such as a keyboard or a mouse connected to the control unit 60. The position G of the center of gravity of the blood vessel may be represented by three-dimensional coordinates.

In addition, the vascular puncture device 11 includes the display unit 70 that displays relative positions of the blood vessel and the distal end of the outer tube 33. As a result, an operator can perform a procedure while confirming the position of the distal end of the outer tube 33 with respect to the blood vessel by the display unit 70, and thus, the procedure can be performed relatively safely and efficiently. The display unit 70 that displays the relative positions of the blood vessel and the distal end of the outer tube 33 is preferably the same as a display unit that displays the cross-sectional image, but may be a different display unit 70.

In addition, the vascular puncture device 11 includes the detection unit 80 capable of detecting the position of the distal end of the outer tube 33 with respect to the distal end of the inner needle 31. As a result, the vascular puncture device 11 detects and uses actual information on the position of the distal end of the outer tube 33 with respect to the distal end of the inner needle 31 so that the distal end of the outer tube 33 can be disposed at a target position (the position G of the center of gravity of the blood vessel) with relatively high accuracy.

In addition, the control unit 60 can receive a detection result from the detection unit 80. As a result, the vascular puncture device 11 can dispose the distal end of the outer tube 33 at the target position with relatively high accuracy based on the detection result from the detection unit 80.

In addition, the vascular puncture device 11 includes the reaction force measurement unit 90 that detects a reaction force acting on the inner needle 31 during puncture. As a result, it is possible to determine a puncture situation by the inner needle 31 from the reaction force, and thus, it is possible to enhance the relatively safety of the vascular puncture device 11.

In addition, the vascular puncture device 11 includes an outer tube holding unit (the second linear movement portion 48) capable of fixing the position of the outer tube 33 independently of the inner needle 31. As a result, it is possible to remove only the inner needle 31 in a state where the outer tube 33 is fixed to suppress removal of the outer tube 33 after the outer tube 33 is disposed at the target position.

In addition, the control unit 60 calculates the movement distance and/or the puncture angle of the drive unit 40 from the information on the cross-sectional image and the separation distance between the distal end of the inner needle 31 and the distal end of the outer tube 33 using a machine-learned model. As a result, the control unit 60 can calculate the movement distance and/or the puncture angle of the drive unit 40 with relatively high accuracy based on a plurality of pieces of stacked data.

The vascular puncture system 10 according to the present embodiment is the vascular puncture system 10 including: the inner needle 31 having the sharp needle tip 32; the flexible outer tube 33 covering the inner needle 31; the drive unit 40 moving the inner needle 31 and the outer tube 33; the imaging unit 22 capable of acquiring a cross-sectional image of a human body in contact with a skin surface and visualizing a distal end of the inner needle 31; and the control unit 60 capable of controlling movement of the drive unit 40. The control unit 60 calculates a movement distance and/or a puncture angle of the drive unit 40 based on information on the cross-sectional image and a separation distance between the distal end of the inner needle 31 and a distal end of the outer tube 33, and moves the drive unit 40 to set the drive unit 40 at the calculated movement distance and/or the calculated puncture angle.

In the vascular puncture system 10 configured as described above, a position of the distal end of the outer tube 33 can be calculated by the control unit 60 when vascular puncture is automatically performed by the inner needle 31, and thus, the distal end of the outer tube 33 covering the inner needle 31 can be appropriately disposed in the blood vessel. Therefore, when the inner needle 31 is removed, it is possible to suppress the distal end of the outer tube 33 from coming out of the blood vessel and to reduce the burden on a patient during re-puncture.

In addition, the drive unit 40 includes the first linear movement portion 42 that linearly moves the inner needle 31 with respect to the outer tube 33 along an extending direction of the inner needle 31, and the second linear movement portion 48 that integrally and linearly moves the inner needle 31 and the outer tube 33 along the extending direction of the inner needle 31. As a result, the inner needle 31 and the outer tube 33 can be integrally moved by the second linear movement portion 48 during puncture. Then, the inner needle 31 can be removed from the outer tube 33 by the first linear movement portion 42.

In addition, the inner needle 31 and the outer tube 33 can be connected to each other. As a result, the inner needle 31 and the outer tube 33 can be integrally moved without changing a distance between the distal end of the inner needle 31 and the distal end of the outer tube 33, and thus, it is possible to suppress deviation of a position of the distal end of the outer tube 33 estimated by the control unit 60.

The present disclosure is not limited to the above-described embodiment, and various modifications can be made by those skilled in the art within the technical idea of the present disclosure. For example, the drive unit 40 can have five movable portions (the first linear movement portion 42, the second linear movement portion 48, the third linear movement portion 45, the rotation portion 46, and the inclination portion 43), but the number of movable portions may be six or more or four or less.

In addition, the position G of the center of gravity of the blood vessel to be punctured is detected from the cross-sectional image in the present embodiment. However, a position other than the position G of the center of gravity of the blood vessel to be punctured may be detected, and the position may be set as a target position at which the distal end of the outer tube 33 is made to arrive. The target position at which the distal end of the outer tube 33 is made to arrive is preferably separated from the front wall toward the back wall of the blood vessel.

In addition, the vascular puncture device 11 or the vascular puncture system 10 may have a function of displaying a blood vessel determined to be punctured or a medical device adapted to a punctured blood vessel. An operator inserts, for example, a sheath along the outer tube 33 after puncturing the blood vessel with the puncture unit 30 and removing the inner needle 31. An outer diameter of the sheath is preferably equal to or smaller than an inner diameter of the blood vessel into which the sheath is to be inserted. This is because when the outer diameter of the sheath is equal to or larger than the inner diameter of the blood vessel, complications are likely to be caused by inserting the sheath into the blood vessel. As an example of a method of calculating the inner diameter of the blood vessel, a length of a diagonal line passing through a center of gravity of an inner peripheral surface of the identified blood vessel (artery or vein) is acquired for the entire circumference at predetermined angle intervals (for example, in the interval of 1 degree), and an average value of the length of the diagonal line passing through the center of gravity of the inner peripheral surface of the identified blood vessel can be set as the inner diameter of the blood vessel. Alternatively, the inner diameter of the blood vessel may be calculated back from an area of the inside of the inner peripheral surface of the blood vessel. When an inner diameter of a blood vessel of an artery, it is preferable to detect the inner diameter of the blood vessel at a certain timing since the artery pulsates. In addition, the certain timing is preferably a timing when the blood vessel contracts most. Since the minimum inner diameter of the inner diameter of the blood vessel is larger than the outer diameter of the medical device to be inserted, the occurrence of complications can be reduced. After calculating the inner diameter of the blood vessel, the control unit 60 can display the outer diameter and a product type of the medical device adapted to the calculated inner diameter of the blood vessel on a display device such as a monitor together with the cross-sectional image. The control unit 60 may identify at least one of an optimal outer diameter, length, or product type of the inner needle 31 from information on a blood vessel determined to be punctured, past statistical information, and the like, display the same on a display device such as a monitor together with a cross-sectional image to present the same to the operator.

In addition, the drive unit 40 may be a robot arm. In addition, the blood vessel to be punctured may be a vein.

The detailed description above describes embodiments of a vascular puncture device and a vascular puncture system capable of detecting and puncturing a position of a blood vessel from an image acquired by an echographic device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A vascular puncture device that punctures a blood vessel, the vascular puncture device comprising:
an inner needle including a needle tip;
an outer tube that is flexible and configured to cover the inner needle;
a drive unit configured to move the inner needle and the outer tube;
an imaging unit configured to acquire a cross-sectional image of a human body in contact with a skin surface and visualize a distal end of the inner needle;
a control unit configured to control movement of the drive unit; and
wherein the control unit is configured to calculate one or more of a movement distance and a puncture angle of the drive unit based on information on the cross-sectional image and a separation distance between the distal end of the inner needle and a distal end of the outer tube, and move the drive unit to set the drive unit at one or more of the calculated movement distance and the calculated puncture angle.

2. The vascular puncture device according to claim 1, wherein the control unit is configured to control the drive unit to make a position, separated by the separation distance in a proximal direction from the distal end of the inner needle along an axial center of the inner needle, coincident with a position of a center of gravity of the blood vessel calculated from the information on the cross-sectional image when puncture of the blood vessel is completed.

3. The vascular puncture device according to claim 1, further comprising:
a display unit configured to display relative positions of the blood vessel and the distal end of the outer tube.

4. The vascular puncture device according to claim 1, further comprising:
a detection unit configured to detect a position of the distal end of the outer tube with respect to the distal end of the inner needle.

5. The vascular puncture device according to claim 4, wherein the control unit is configured to receive a detection result from the detection unit.

6. The vascular puncture device according to claim 1, further comprising:
a reaction force measurement unit configured to detect a reaction force acting on the inner needle during puncture.

7. The vascular puncture device according to claim 1, further comprising:
an outer tube holding unit configured to fix a position of the outer tube independently of the inner needle.

8. The vascular puncture device according to claim 1, wherein the control unit includes a machine-learned model configured to receive inputs of the information on the cross-sectional image and the separation distance between the distal end of the inner needle and the distal end of the outer tube and output the movement distance and/or the puncture angle of the drive unit.

9. A vascular puncture system comprising:
an inner needle including a needle tip;
an outer tube that is flexible and configured to cover the inner needle;
a drive unit configured to move the inner needle and the outer tube;
an imaging unit configured to acquire a cross-sectional image of a human body in contact with a skin surface and visualize a distal end of the inner needle;
a control unit configured to control movement of the drive unit; and
wherein the control unit is configured to calculate one or more of a movement distance and a puncture angle of the drive unit based on information on the cross-sectional image and a separation distance between the distal end of the inner needle and a distal end of the outer tube, and move the drive unit to set the drive unit at one or more of the calculated movement distance and the calculated puncture angle.

10. The vascular puncture system according to claim 9, wherein the drive unit includes:
a first linear movement portion configured to linearly move the inner needle with respect to the outer tube along an extending direction of the inner needle; and
a second linear movement portion configured to integrally and linearly move the inner needle and the outer tube along the extending direction of the inner needle.

11. The vascular puncture system according to claim 9, wherein the inner needle and the outer tube are connectable to each other.

12. The vascular puncture system according to claim 9, wherein the control unit is configured to control the drive unit to make a position, separated by the separation distance in a proximal direction from the distal end of the inner needle along an axial center of the inner needle, coincident with a position of a center of gravity of the blood vessel calculated from the information on the cross-sectional image when puncture of the blood vessel is completed.

13. A method for puncturing a blood vessel, the method comprising:
acquiring a cross-sectional image of a human body in contact with a skin surface and visualizing a distal end of an inner needle, the inner needle including a needle tip;
calculating one or more of a movement distance and a puncture angle based on information on the cross-sectional image and a separation distance between the distal end of the inner needle and a distal end of an outer tube, the outer tube covering the inner needle; and
setting a drive unit at one or more of the calculated movement distance and the calculated puncture angle, the drive unit configured to move the inner needle and the outer tube.

14. The method according to claim 13, further comprising:
controlling the drive unit to make a position, separated by the separation distance in a proximal direction from the distal end of the inner needle along an axial center of the inner needle, coincident with a position of a center of gravity of the blood vessel calculated from the information on the cross-sectional image when puncture of the blood vessel is completed.

15. The method according to claim 13, further comprising:
displaying relative positions of the blood vessel and the distal end of the outer tube.

16. The method according to claim 13, further comprising:
detecting a position of the distal end of the outer tube with respect to the distal end of the inner needle.

17. The method according to claim 16, further comprising:
receiving a detection result.

18. The method according to claim 14, further comprising:
detecting a reaction force acting on the inner needle during puncture.

19. The method according to claim 14, further comprising:
fixing a position of the outer tube independently of the inner needle.

20. The method according to claim 14, further comprising:
receiving inputs of the information on the cross-sectional image and the separation distance between the distal end of the inner needle and the distal end of the outer tube and outputting one or more of the movement distance and the puncture angle of the drive unit using a machine-learned model.
